(19) United States
(12) Patent Application Publication
(10) Pub. No.: US 2026/0124451 A1
(43) Pub. Date: May 7, 2026

(54) SYSTEM FOR PAIN MANAGEMENT UTILIZING RESORBABLE ELECTRODES

(71) Applicant: Becton, Dickinson and Company, Franklin Lakes, NJ (US)

(72) Inventors: Leandro Forciniti, Haskell, NJ (US); Georgios Yiorgos Bertos, Chicago, IL (US); Anne Shim, Ringwood, NJ (US)

(21) Appl. No.: 18/934,323

(22) Filed: Nov. 1, 2024

(51) Int. Cl. A61N 1/36; A61N 1/05; A61N 1/372

(52) U.S. Cl. CPC A61N 1/36071; A61N 1/0556; A61N 1/36146; A61N 1/37223; A61N 1/37247

(57) ABSTRACT

A pain management system includes: a resorbable electrode mounted on a substrate configured to be implanted on the nerve bundle of a patient, the resorbable electrode being configured to emit electrical pulses to the nerve bundle; and an electrical signal transmitter operatively and wirelessly connected with the resorbable electrode, the electrical signal transmitter configured to transmit signals to the electrode to generate electrical pulses, the electrical signal transmitter configured to be mounted on the patient's skin near the resorbable electrode.

### FIELD OF THE INVENTION

The present invention is directed generally to pain management, and more particularly to non-pharmaceutical pain management strategies and devices.

### BACKGROUND

Opioids are very commonly used for pain management of patients, particularly after a traumatic event (e.g., a surgical procedure). However, opioids can be very addictive. In the US, studies have shown that 3 million surgical American patients continued to take opioid drugs for three to six months after their initial exposure to drugs following surgery, and 10 percent of such patients transitioned to long-term opioid use after surgery (which is likely to cause addiction).

Because there is a significant effort to minimize hospital stays for recovering surgical patients to reduce medical costs, many surgical patients are sent home to recuperate, with family members or other caretakers left to supervise or assist with the recovery effort. This often includes the distribution of medications to the patient, which may be both medications to assist in recovery as well as pain management medications. Particularly common surgeries of this type include total knee replacement, total hip replacement, rotator cuff, colectomy, sleeve gastrectomy, hernia, and hysterectomy. As one example, a typical rotator cuff surgery may require the caretaker to manage a TENS unit that provides electrical stimulation, a cooling sleeve (e.g., a “Cryo-Cuff”) connected to an ice bath that requires periodic refilling, a pharmaceutical slow-release nerve block, and opioids as needed.

To save the caretakers time and effort, some physicians will “overprescribe” pain medications (such as opioids) to ensure that the patient has a sufficient supply. However, overprescribing pain pills after surgery led to a surplus of 3.3 billion unused opioids in 2016, a glut of extra drugs that likely contributed to the United States'burgeoning opioid crisis. This oversupply has doubtless impacted tens of thousands of opioid overdose deaths in the U.S.

In view of the foregoing, it would be desirable to provide alternative pain relief protocols, and in particular non-pharmaceutical pain relief protocols.

### SUMMARY

As a first aspect, embodiments of the invention are directed to a pain management system. The pain management system comprises: a resorbable electrode mounted on a substrate configured to be implanted on the nerve bundle of a patient, the resorbable electrode being configured to emit electrical pulses to the nerve bundle; and an electrical signal transmitter operatively and wirelessly connected with the resorbable electrode, the electrical signal transmitter configured to transmit signals to the electrode to generate electrical pulses, the electrical signal transmitter configured to be mounted on the patient's skin near the resorbable electrode.

As a second aspect, embodiments of the invention are directed to a method of managing pain for a patient in need of such treatment. The method comprises wirelessly transmitting electrical signals from an electrical signal transmitter mounted on the skin of the patient to a resorbable electrode applied to a nerve bundle of the patient, wherein the transmitting of electrical signals causes the resorbable electrode to emit electrical pulses to the nerve bundle.

As a third aspect, embodiments of the invention are directed to a pain management system comprising: a cuff comprising a resorbable electrode mounted on a resorbable substrate configured to be implanted on the nerve bundle of a patient, the resorbable electrode being configured to emit electrical pulses to the nerve bundle; a medical device configured to be mounted on the skin of a patient near the resorbable electrode; and an electrical signal transmitter operatively and wirelessly connected with the resorbable electrode, the electrical signal transmitter configured to transmit signals to the electrode to generate electrical pulses, the electrical signal transmitter mounted on the medical device.

### BRIEF DESCRIPTION OF THE FIGURES

FIG. 1A is a schematic perspective view of a pain management cuff according to embodiments of the invention.

FIG. 1B is a schematic perspective view of the pain management cuff of FIG. 1A that shows the flexible nature of the cuff.

FIG. 2 is a schematic perspective view of the pain management cuff of FIG. 1A implanted on a nerve bundle.

FIG. 3 is a schematic diagram of a pain management system according to embodiments of the invention that utilizes a pain management cuff such as that of FIG. 2.

FIGA. 4A-4D are schematic views of different electrodes that may be used with the pain management cuff of FIG. 1A.

FIG. 5 is a catheter securement assembly that includes a signal transmitter as in FIG. 3 to excite a pain management cuff implanted on a nerve bundle as in FIG. 2.

FIG. 6 is a schematic diagram illustrating the components of the signal transmitter shown in FIG. 5.

FIG. 7 is a schematic diagram of a pain management system according to further embodiments of the invention, wherein the signal transmitter is controlled by an external device such as a cellular phone.

### DETAILED DESCRIPTION

The present invention will now be described more fully hereinafter, in which preferred embodiments of the invention are shown. This invention may, however, be embodied in different forms and should not be construed as limited to the embodiments set forth herein. Rather, these embodiments are provided so that this disclosure will be thorough and complete, and will fully convey the scope of the invention to those skilled in the art. In the drawings, like numbers refer to like elements throughout. Thicknesses and dimensions of some components may be exaggerated for clarity.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein the expression “and/or” includes any and all combinations of one or more of the associated listed items.

In addition, spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Also, as used herein, the terms “cap” and “closure” are used interchangeably to refer to a component that caps or closes a pharmaceutical vial.

Well-known functions or constructions may not be described in detail for brevity and/or clarity.

One non-pharmaceutical pain relief technique that shows promise is the application of targeted electrical stimulation to the patient's nervous system. Electrical stimulation is typically classified into several forms depending on the configuration of the electrodes relative to the targeted nerve. Transcutaneous electrical nerve stimulation (TENS) relies on electric stimuli (i.e., biphasic pulses with amplitudes of 1 to 60 mA, durations of 50 to 200 μs, and frequencies of 1 to 200 Hz) delivered through patch electrodes placed on the skin near the source of pain. The resulting electrical paresthesia alleviates pain by distracting the brain from painful stimuli. Although this form of electrotherapy is noninvasive, the large distances between the electrodes and the targeted nerves limit the efficacy due to the insulating effects of the skin and muscle/fat interlayers. Percutaneous electrical nerve stimulation (PENS) overcomes this disadvantage through the use of electrodes that penetrate the skin to deliver electrical stimuli (i.e., biphasic pulses with amplitudes of 0.2 to 20 mA, durations of 15 to 200 μs, and frequencies of 2 to 100 Hz) in proximity to the nerves. This method can effectively block nerve conduction, but temporary pain and tissue disruption can occur during and after electrode insertion. In addition, inaccurate positioning can lead to damage to the nerve itself.

To date, perhaps the most effective approach uses electrodes in cylindrical cuff geometries to deliver kilohertz-frequency alternating current (KHFAC) (i.e., sinusoidal waveforms with an amplitude of 10 Vₚₚ and frequencies of 25 to 35 kHz as a efficacious condition) directly to the peripheral nerves. The KHFAC stimulation maintains dynamic steady-state depolarization, thereby arresting action potentials in the axons that carry pain signals when they reach the depolarizing charge field. This scheme is attractive relative to TENS or PENS because it can reliably and completely block nerve conduction with response times less than 10 ms that are much faster than these alternatives (seconds). A major disadvantage of conventional cuff electrodes for KHFAC stimulation, however, lies in complex surgical procedures necessary for implantation and, if necessary, for subsequent removal, with associated risks for additional pain and discomfort, and possible permanent damage to the nerve. In addition, implantation of nonbioresorbable, stiff nerve cuffs can lead to inflammatory cell infiltration and morphological changes (e.g., fibrous capsule) to the nerve as part of the mechanisms that protect nerve functionality from foreign body reactions. These processes result in difficulties in safe removal of a cuff electrode from the nerve.

To address some of the shortcomings of stimulation via cylindrical cuffs, a bioresorbable peripheral nerve stimulator has been developed. One such device is described in Lee, A bioresorbable peripheral nerve stimulator for electronic pain block, Science Advances (Vol. 8, No. 40). This device comprises a thin, flexible bioresorbable cuff for KHFAC nerve block, wherein direct electrode-nerve contact enables complete, fast operation and natural processes of bioresorption to eliminate the need for surgical removal. Key advances relative to related bioresorbable nerve stimulators for neuroregeneration include (i) multimaterial structures (e.g., a PGLA (poly(lactic-co-glycolic acid)) or PA (polyanhydride) base with molybdenum and/or magnesium electrodes) that support high-voltage, high-frequency stimulation over extended time periods, aligned to clinical needs for pain mitigation; (ii) bioresorbable cuffs that exploit woven structure geometries for reliable delivery of KHFAC; (iii) schemes for electrical and mechanical decoupling between the cuff and extension electrodes by selective bioresorption to reduce the potential for damage to the nerve; and (iv) demonstrations of complete, fast, and reversible electronic nerve conduction block in live animal models. These results establish the basis for a temporary, bioelectronic form of medicine for pain management, with potential to replace or complement traditional pharmacological schemes and conventional electrical stimulation approaches.

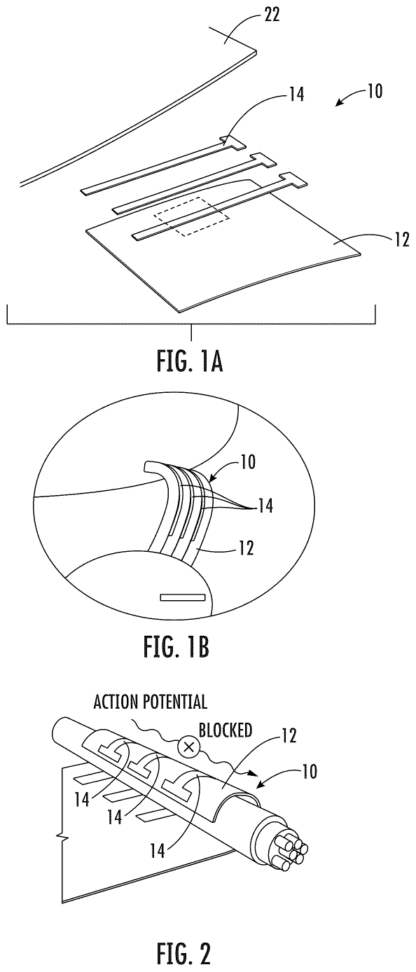

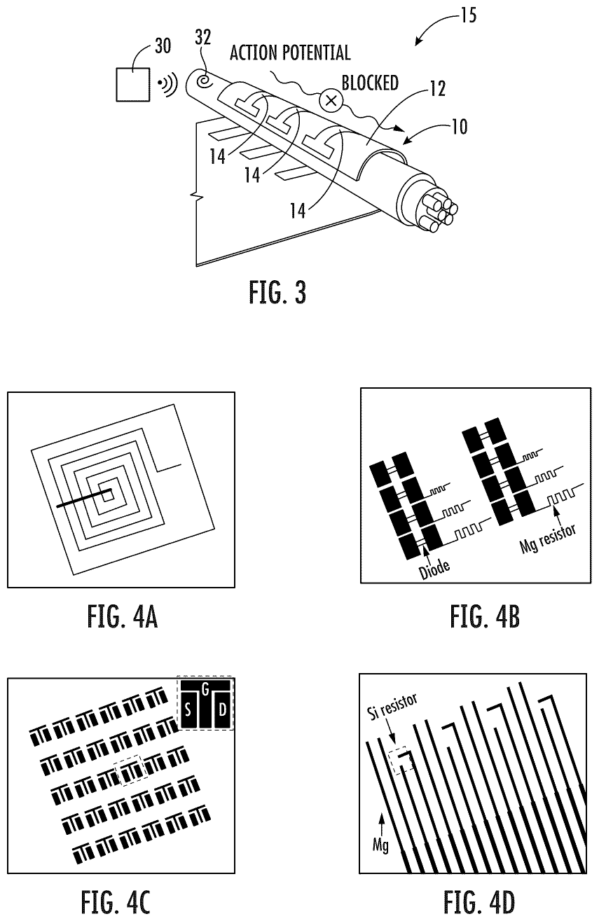

One exemplary cuff is shown in FIGS. 1A and 1B. The cuff 10 (shown in a flat configuration for clarity, with the understanding that it would be rolled into a sleeve to encase a nerve bundle) has a base 12 comprising PLGA, electrodes 14 (formed of molybdenum (Mo) in this instance) that are arranged in parallel segments on the base 12 and covered with conductive wax, and a top layer 22 formed of PA. Electrodes of Mo in a cuff structure form a stable interface to the targeted nerve during delivery of blocking stimuli. The thermoplastic nature of PLGA and its low glass transition temperature (T_{g}: 40 to 60° C.) provide a simple means for thermally joining the overlapping portion in the PLGA support through local heating (˜60° C.) applied carefully during implantation in a manner that avoids damage to the nerve. In this way, the cuff can be wrapped circumferentially around the nerve to ensure conformal, direct contact (see FIG. 2). The PA encapsulant serves as a bioresorbable barrier with superior properties compared to those of PLGA to prevent interaction of biofluids with the electrode traces. The resulting cuff 10 (e.g., 8 mm wide, 400 μm thick) can offer thin, flexible mechanical properties that allow the structure to adapt to natural movements after implantation. The aforementioned materials have been shown in gradually resorb in the patient, eliminating the need for any subsequent procedure to remove them.

Once implanted in a patient encircling a nerve bundle as in FIG. 2, the cuff 10 can be energized to generate the electrical stimulation pulses capable of blocking pain. The electrodes 14 may deliver electrical stimulation pulses that vary in frequency, amplitude and pulse width as needed. Exemplary ranges include a frequency of between about 0.001 and 5,000,000 kHz (wherein a range of 1,000 to 1,000,000 kHz may be used in some embodiments), an amplitude of 0.001 to 1,000 mAmps, and pulse duration of 0.001 to 10,000 μs.

The base 12, top layer 22 and electrodes 14 of the cuff 10 may be formed of any resorbable materials suitable for the generation of electrical pulses. Exemplary electrode materials include conducting polymers such as polypyrrole, polyaniline, poly(3,4-ethylenedioxythiophene), and conductively-filled base polymers that comprise a base resin including a biocompatible polymer such as high density polyethylene, polyurethane elastomers, polyvinylchloride, polypropylene, medical-grade silicone, collagen hyaluronic acid, and a conductive filler that is loaded into the base polymer. Conductive fillers may include conductive carbons such as acetylene black, channel black, furnace black, lamp black, thermal black, graphene, and carbon nano-tubes (both single wall and multiwall types) or a class of other conductive fillers such as silver, gold, copper, iron and their conductive oxides. Alternately, the exemplary electrode materials can be made of printed copper, gold or silver materials or metallic tapes. Exemplary materials for the base 12 and/or the top layer 22 include polyurethane elastomers, polypropylene, polyethylene terephthalate, polyethylene glycol, medical-grade silicone, co-polyesters, poly(vinyl)alcohol, polylactic acid, polycaprolactone, polyhydroxyalkanoates, chitosan, and polybutylene succinate, and can include any materials that can provide a suitable resorbable mounting substrate for the electrodes 14. In some embodiments, the cuff may resorb in between about 30 and 180 days.

To date, experimentation on live subjects has focused on animal studies, wherein the electrodes are connected with an electrical signal source that is hard-wired to the electrodes. However, in practice with human patients, including hard-wiring between the signal source and the electrodes would somewhat defeat the purpose of using a resorbable sleeve, as presumably the wiring would need to be removed.

To address this problem, and according to embodiments of the invention, a wireless signal transmitter may be employed to generate a signal in the electrodes. A schematic diagram shown in FIG. 3 illustrates the general structure of a pain management system 15. A signal transmitter 30 located outside of the patient's body generates signals in the electrodes 14 implanted in the patient's body (e.g., wrapped around a nerve bundle). The electrical impulses generated in the electrodes 14 provide pain relief for the patient in the manner described above. In some embodiments, the signal transmitter 30 may be a transmitter coil, which can generate electrical pulses in the electrodes 14 via induction through magnetic coupling tied to a receiver coil 32 mounted on the base 12, although other methods of generating electrical impulses may also be suitable.

FIGS. 4A-4D illustrate additional types and arrangements of electrodes and patterns that may be employed in a pain management cuff 10.

When a signal transmitter 30 that generates impulses in the electrodes 14 based on induction is employed, it may be desirable that the signal transmitter is near the electrodes 14, and that it remain generally stationary relative to the electrodes 14, as a nearby, fixed signal transmitter 30 can provide reliable signals to the electrodes 14. One technique for ensuring that the transmitter 30 remains both fixed and near the electrodes 14 is to incorporate the transmitter 30 into a medical device, component, etc. that is associated with the procedure that results in the need for pain management.

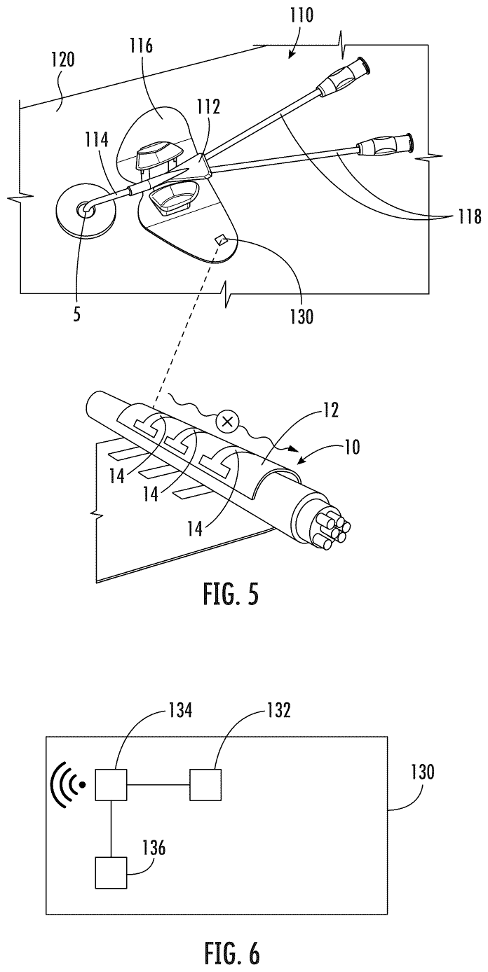

As one example of this concept, FIG. 5 shows a catheter assembly designated broadly at 110. The catheter assembly 110 includes a catheter hub 112 that feeds an attached catheter 114 that is inserted into an insertion site S on the patient's skin. The catheter hub 112 is fed by one or more inlet lines 118. The catheter hub 112 is mounted on a securement device 116 that is adhered to the skin adjacent the insertion site S (an exemplary securement device 116 is the STATLOCK™ device, available from Becton, Dickinson and Co. (Franklin Lakes, New Jersey)). The securement device 116 maintains the catheter hub 112, and in turn the catheter 114, in position at the insertion site S. A dressing 119 protects the insertion site S.

As shown in FIG. 5, in embodiments of the invention, the securement device 116 may include a signal transmitter 130 as described above that is operatively and wirelessly connected with the electrodes 14 of the cuff 10. A sleeve or cuff 10 as described above is applied to the appropriate nerve bundle that produces pain to be managed with the cuff 10 (which is often, but need not be, closely adjacent to the signal transmitter 130). Thus, once the cuff 10 is applied, the wound is closed, and the securement device 116 with its signal transmitter 130 is mounted on the patient's skin near the cuff 10 (e.g., within 1 to 500 mm of the cuff 10). Signals can then be transmitted wirelessly from the signal transmitter 130 to the electrodes 14 of the cuff 10 to induce the generation of electrical pulses that can assist with pain management.

Because it is typically undesirable to frequently change the catheter 114, the securement device 116 typically remains in place for several days. As such, it may be a very suitable device on which to mount the transmitter 130, as it will remain for quite some time in a location near the nerve bundle around which the cuff 10 is wrapped. Once either pain is absent or the cuff 10 is resorbed, the securement device 116 can be removed.

The signal transmitter 130 (FIG. 6) may be any type of transmitter that can provide wireless signals to the electrodes 14 of the cuff 10 to produce the desired therapeutic electrical pulses. Such a transmitter 130 may include a battery 132 or other power device to provide power to the transmitter 130. The transmitter 130 may also include a signal generator 134 to generate the desired signals. Further, the transmitter 130 may include a control device 136 that can modify the signals produced by the signal generator 134 (e.g., frequency, amplitude, and/or pulse duration) as desired or needed for pain management. The transmitter 130 may include other devices or components as well.

Those of skill in this art will appreciate that, although the signal transmitter 130 is shown herein as mounted in the securement device 116, in other embodiments the signal transmitter 130 may be mounted on a different medical device. For example, in some embodiments the signal transmitter 130 may be mounted on the catheter hub 112, or the catheter 114, or even on the dressing 119, as long as the device of choice is located (and typically affixed to the patient's skin) at a distance that enables the electrodes 14 of the cuff 10 to receive the signals from the transmitter 130. Other exemplary devices that may be used for mounting of the signal transmitter 130 include ECG electrodes, pulse oximeters, patient wrist bands, consumer fitness watches or devices, syringe infusion systems, and insulin pumps.

Exemplary medical procedures in which a patient may benefit from the utilization of the pain management system 15 include surgeries (e.g., rotator cuff, knee replacement, hip replacement, ankle replacement, spinal fusion, cardiac surgeries, hemodialysis placement, caesarean, long-term intravenous therapy through port access, and general trauma, in which a nerve bundle is located sufficiently near the surgical site that pain can occur due to the trauma of the procedure. Other types of medical procedures that may provide pain relief to the patient include nerve blocks, steroid injections, patient-controlled analgesia, radiofrequency ablation, basivertebral nerve ablation, botulinum toxin injection and epidural steroid injections.

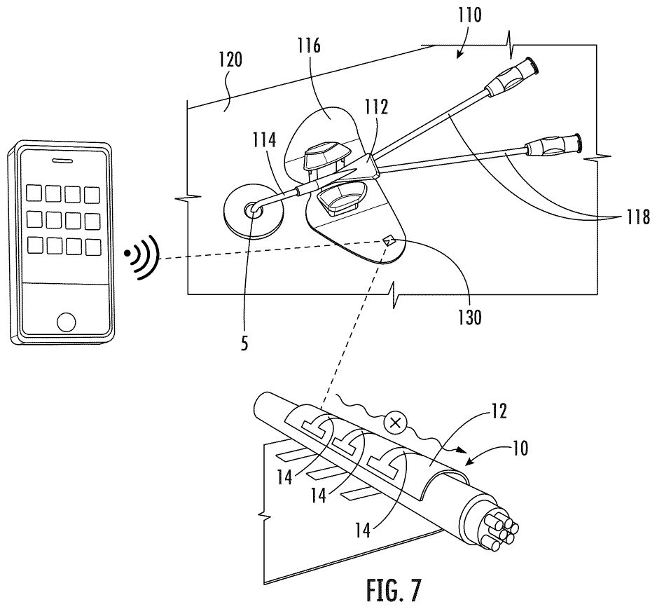

Further, although the signal transmitter 130 is illustrated in FIG. 5 as a stand-alone device, in some embodiments the signal transmitter 130 may be driven or controlled by an external driving device (e.g., a tablet, a “smart” phone or similar handheld device, or a dedicated device that is specific to the signal transmitter 130). Such an external driving device is shown schematically in FIG. 7 at 250. The driving device 250 communicates with the signal transmitter 130 to control its operation. For example, the driving device 250 may have the ability to activate and/or deactivate the signal transmitter 130 (e.g., through an “app” that is downloaded onto the “smart” phone).

As another example, the driving device 250 may be able to communicate with the signal transmitter 130 to vary the signals transmitted from the signal transmitter 130, such that the signals transmitted to the electrodes 14 can be varied in character (e.g., to vary one or more of the pulse width, frequency, and amplitude) as the patient desires for managing pain. In this manner the system can mimic an IV-based “pain drip” that allows a patient to control the level of pain medication as needed.

In addition, in some embodiments the signal transmitter 130 may include a sensor that detects feedback signals from the electrodes 14 and/or from the nerve itself. Such feedback may be useful to determine the effect or impact of the electrical pulses emanating from the electrodes 14 and to adjust the signals being transmitted accordingly.

As a still further embodiment, resorbable electrodes and associated signal transmitters may be employed to assist in the regeneration of peripheral nerve tissue. In such an arrangement, cuffs 10 may be implanted at two endpoints on a damaged or unresponsive nerve. Transmission of signals between the two endpoints may “kick-start” or otherwise enhance the regeneration of nerve tissue that has lost the ability to feel pain.

The foregoing is illustrative of the present invention and is not to be construed as limiting thereof. Although exemplary embodiments of this invention have been described, those skilled in the art will readily appreciate that many modifications are possible in the exemplary embodiments without materially departing from the novel teachings and advantages of this invention. Accordingly, all such modifications are intended to be included within the scope of this invention as defined in the claims. The invention is defined by the following claims, with equivalents of the claims to be included therein.

What is claimed is:

1. A pain management system, comprising:
a resorbable electrode mounted on a substrate configured to be implanted on the nerve bundle of a patient, the resorbable electrode being configured to emit electrical pulses to the nerve bundle; and
an electrical signal transmitter operatively and wirelessly connected with the resorbable electrode, the electrical signal transmitter configured to transmit signals to the electrode to generate electrical pulses, the electrical signal transmitter configured to be mounted on the patient's skin near the resorbable electrode.

2. The pain management system defined in claim 1, wherein the electrical signal transmitter is configured to control at least one of pulse width, amplitude and frequency of electrical pulses emitted from the resorbable electrode.

3. The pain management system defined in claim 1, further comprising a medical device configured to be mounted to the skin of the patient, and wherein the electrical signal transmitter is mounted in or on the medical component.

4. The pain management system defined in claim 3, wherein the medical device is a component of a catheter securement assembly.

5. The pain management system defined in claim 4, wherein the medical device is selected from the group consisting of: a securement device, a catheter, a catheter hub, and a wound dressing.

6. The pain management system defined in claim 1, further comprising a user interface that is operatively connected with the electrical signal transmitter.

7. The pain management system defined in claim 1, wherein the user interface comprises a cellular telephone that is wirelessly connected with the electrical signal transmitter.

8. The pain management system defined in claim 1, wherein the resorbable electrode is mounted to a cuff, the cuff configured to encircle the nerve bundle.

9. The pain management system defined in claim 8, wherein the electrode is configured to resorb in between about 30 and 180 days.

10. The pain management system defined in claim 1, wherein the electrical signal transmitter is configured to be mounted within about 1 and 500 mm of the resorbable electrode.

11. The pain management system defined in claim 1, wherein the electrical signal transmitter is configured to generate signals in the resorbable electrode via induction.

12. A method of managing pain for a patient in need of such treatment, comprising wirelessly transmitting electrical signals from an electrical signal transmitter mounted on the skin of the patient to a resorbable electrode applied to a nerve bundle of the patient, wherein the transmitting of electrical signals causes the resorbable electrode to emit electrical pulses to the nerve bundle.

13. The method defined in claim 12, wherein the electrical signals control at least one of pulse width, amplitude and frequency of the electrical pulses emitted from the resorbable electrode.

14. The method defined in claim 12, wherein the transmitter is mounted on a medical device that is mounted on the skin of the patient.

15. The method defined in claim 14, wherein the medical device is a component of a catheter securement assembly.

16. The method defined in claim 15, wherein the medical device is selected from the group consisting of: a securement device, a catheter, a catheter hub, and a wound dressing.

17. The method defined in claim 12, wherein the transmitter is operatively connected with a user interface, and wherein manipulation of the user interface controls the character of the electrical pulses emitted from the resorbable electrode.

18. The method defined in claim 17, wherein the user interface comprises a cellular telephone that is wirelessly connected with the electrical signal transmitter.

19. The method defined in claim 12, wherein the resorbable electrode is mounted to a cuff that encircles the nerve bundle.

20. The method defined in claim 12, wherein the electrical signal transmitter is mounted within about 1 and 500 mm of the resorbable electrode.

21. The method defined in claim 12, wherein the electrical signal transmitter generates signals in the resorbable electrode via induction.

22. The method defined in claim 12, carried out for between about 30 and 180 days.

23. A pain management system, comprising:
a cuff comprising a resorbable electrode mounted on a resorbable substrate configured to be implanted on the nerve bundle of a patient, the resorbable electrode being configured to emit electrical pulses to the nerve bundle;
a medical device configured to be mounted on the skin of a patient near the resorbable electrode; and
an electrical signal transmitter operatively and wirelessly connected with the resorbable electrode, the electrical signal transmitter configured to transmit signals to the electrode to generate electrical pulses, the electrical signal transmitter mounted on the medical device.

24. The pain management system defined in claim 23, wherein the medical device is selected from the group consisting of: a securement device, a catheter, a catheter hub, and a wound dressing.
